(19) United States
(12) Patent Application Publication
(10) Pub. No.: US 2007/0219223 A1
(43) Pub. Date: Sep. 20, 2007

(54) Compositions and methods for treating respiratory disorders

(75) Inventors: Constance N. Wilson, Raleigh, NC (US); John J. Partridge, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Endacea, Inc., Research Triangle Park, NC (US)

(21) Appl. No.: 11/715,089

(22) Filed: Mar. 7, 2007

Related U.S. Application Data
(60) Provisional application No. 60/779,722, filed on Mar. 7, 2006.

(51) Int. Cl. A61K 31/522; C07D 473/04

(52) U.S. Cl. 514/263.35; 544/272

(57) ABSTRACT

Methods and compositions for treating and preventing respiratory disorders are provided. The methods of the invention comprise administering to a subject a therapeutically effective amount of a pharmaceutically acceptable salt of an A₁ adenosine receptor antagonist, particularly 1-hydroxy-2-naphthoic acid salts or 3-hydroxy-2-naphthoic acid salts, more particularly a 1-hydroxy-2-naphthoic acid salt of 3-[2-(4-aminophenyl)ethyl]-8-benzyl-7-{2-[ethyl-(2-hydroxyethyl)amino]ethyl}-1-propyl-3,7-dihydropurine-2,6-dione (i.e., an L-97-1 xinafoic acid salt). Hydrates of the A₁ adenosine receptor antagonist salts described herein are further provided. The invention further encompasses pharmaceutical compositions comprising a pharmaceutically acceptable salt of an A₁ adenosine receptor antagonist in a pharmaceutically acceptable carrier. The compositions of the invention find use in methods for treating and preventing respiratory disorders.

### CROSS REFERENCE TO RELATED APPLICATIONS

This application claims the benefit of U.S. Provisional Application No. 60/779,722, filed on Mar. 7, 2006, which is herein incorporated by reference in its entirety.

### FIELD OF THE INVENTION

The present invention relates to compositions and methods for the treatment and prevention of respiratory disorders, including allergic rhinitis (i.e., hay fever) and diseases associated with reversible airway obstruction such as asthma.

### BACKGROUND OF THE INVENTION

Asthma is a chronic lung condition that affects nearly 20 million Americans and may be classified as allergic (intrinsic) or non-allergic (extrinsic). Patients with asthma experience difficulty breathing as a result of narrowing or obstruction of the airway, making it more difficult to move air in and out. This narrowing can result from airway inflammation and bronchoconstriction, thereby producing the characteristic symptoms of asthma such as wheezing, coughing, shortness of breath, and chest tightness. Although important advancements in the treatment of asthma have been made, much research still needs to be done to offer more effective treatments, particularly pulmonary inhalation therapies, for asthmatic patients.

Allergic rhinitis or “hay fever” involves an allergic reaction to pollen from grasses, trees, and weeds. When pollen is inhaled by an individual suffering from allergic rhinitis, antibody production and histamine release is triggered. Symptoms of allergic rhinitis include but are not limited to coughing, headache, itching of the eyes, mouth, throat, or nose, sneezing, nasal congestion, wheezing, sore throat, and watery eyes. The symptoms associated with hay fever vary significantly from person to person, and allergic rhinitis may be associated with other conditions such as asthma.

Xanthines, including theophylline and bamiphylline, and beta-2 adrenergic receptor agonists, such as salmeterol, produce beneficial bronchodilating effects in asthmatic patients. See, for example, Barnes (2003) Am. J. Resp. Crit. Care Med. 167:813-818; Ginesu et al. (1987) Italian J. Chest Dis. 41:311-316; Goodman & Gilman 's The Pharmacological Basis of Therapeutics, 10^{th} edition (eds. Hardman et al.; McGraw-Hill Publishing Company, New York, 2001). Both classes of drugs, however, have significant adverse side effects. For example, high plasma levels following high oral doses of xanthines are associated with cardiovascular and central nervous system side effects in humans, including increased heart rate and blood pressure, sleeplessness, tremors, and seizures. See Goodman & Gilman, supra. Moreover, following high inhalational doses of beta-2 adrenergic receptor agonists, high plasma levels of the drug have been shown to cause systemic cardiovascular and central nervous system side effects such as increased heart rate, blood pressure, sleeplessness, tremors, and seizures. Id. In part, the side effects of beta-2 adrenergic receptor agonists following inhalational administration may be related to oral absorption of the swallowed portion of the dose, as over half of the dose delivered by oral inhalation is swallowed.

Efforts to reduce the negative side effects associated with asthma medications, particularly those delivered by pulmonary inhalational methods, have been undertaken. For example, in an attempt to reduce oral absorption following inhalational delivery, salmeterol has been formulated as salmeterol xinafoate (salmeterol 1-hydroxy-2-napthoic acid salt), an ionic salt that is sparingly soluble in water and thus has reduced oral absorption. See The Merck Index: An Encyclopedia of Chemicals, Drugs, and Biologicals, 14^{th} edition (O'Neil et al., eds.; Merck Research Laboratories Publishing Division, Whitehouse, N.J., 2006, 8337); and Brogden and Faulds (1991) Drugs, 42(5): 895-912 (and references therein), all of which are herein incorporated by reference in their entirety. Following inhalational delivery, salmeterol xinafoate has a slow onset and long lasting bronchodilating effect, as well as a low rate of adverse side effects. See Groeben and Emala (1999) Chest 115:1678-1683. Thus, formulation of the xinafoate salt of the inhaled bronchodilator salmeterol results in an improved drug efficacy and safety profile.

3-[2-(4-aminophenyl)ethyl]-8-benzyl-7-{2-[ethyl-(2-hydroxyethyl)amino]ethyl}-1-propyl-3,7-dihydropurine-2,6-dione (also referred to herein as L-97-1) and pharmaceutical compositions and pharmaceutically acceptable salts thereof are disclosed and claimed in U.S. Pat. No. 5,786,360 and U.S. Pat. No. 6,489,332. Oral administration of L-97-1 dihydrochloride has been shown to block allergic airway responses in an allergic rabbit model. Obiefuna et al. (2005) J. Pharmacol. Exp. Ther. 315: 329-336. Moreover, inhalational delivery of L-97-1 blocked bronchoconstrictor responses to adenosine in allergic rabbits. L-97-1 dihydrochloride, however, is a xanthine and in high oral or intravenous doses may cause central nervous system side effects, such as seizures, as described above. Chemical modification of L-97-1 to a salt form that has a more favorable systemic side effect profile may augment the use of this drug for the pulmonary inhalational treatment of asthma and other respiratory disorders, including allergic rhinitis, chronic bronchitis, chronic obstructive pulmonary disease, cystic fibrosis, adult respiratory distress syndrome, acute lung injury associated with septicemia or reperfusion organ injury, pulmonary fibrosis, bronchopulmonary dysplasia, emphysema, bronchiolitis obliterans (or bronchiolitis obliterans syndrome), and airway remodeling.

Given the large number of patients afflicted with asthma and other respiratory disorders, a need exists in the art for safer and more effective compositions, particularly compositions suitable for inhalational therapy, and methods for the treatment of these conditions. Such compositions would ideally display reduced systemic side effects and a prolonged duration of action in the lung as a bronchodilator, anti-inflammatory, antimucolytic, antitussive, antifibrotic agent, antiangiogenesis agent, and/or enhancer of airway hydration.

### BRIEF SUMMARY OF THE INVENTION

Compositions and methods for treating respiratory disorders, including allergic rhinitis and asthma, are provided. The compositions of the invention comprise pharmaceutically acceptable salts of A₁ adenosine receptor antagonists, particularly a xinafoic acid salt of L-97-1 (i.e., 3-[2-(4-aminophenyl)ethyl]-8-benzyl-7-{2-[ethyl-(2-hydroxyethyl)amino]ethyl}-1-propyl-3,7-dihydropurine-2,6-dione). The compositions of the invention also comprise other pharmaceutically acceptable salts of A₁ adenosine receptor antagonists, particularly a 3-hydroxy-2-naphthoic acid salt of L-97-1. A pharmaceutical composition comprising a pharmaceutically acceptable salt of an A₁ adenosine receptor antagonist in a pharmaceutically acceptable carrier is also provided. The compositions of the invention find particular use in methods for treating and preventing respiratory disorders, particularly by pulmonary inhalational administration of the composition.

The methods of the invention are directed to treating or preventing a respiratory disorder, particularly diseases associated with reversible airway obstruction, more particularly asthma, comprising administering to a subject a therapeutically effective amount of at least one pharmaceutically acceptable salt of an A₁ adenosine receptor antagonist. In certain embodiments, the salt is selected from the group consisting of an L-97-1 monoxinafoic acid salt (i.e., monoxinafoate salt; a 1:1 salt of L-97-1 and xinafoic acid), an L-97-1 dixinafoic acid salt (i.e., dixinafoate salt; a 1:2 salt of L-97-1 and xinafoic acid), an L-97-1 0.5 xinafoic acid salt (i.e., 0.5 xinafoate salt; a 2:1 salt of L-97-1 and xinafoic acid), and an L-97-1 1.5 xinafoic acid salt (i.e., 1.5 xinafoate salt; a 2:3 salt of L-97-1 and xinafoic acid). In still other embodiments, a salt of the invention is an L-97-1 mono(3-hydroxy-2-naphthoic acid) salt (i.e., mono(3-hydroxy-2-naphthoate salt); a 1:1 salt of L-97-1 and 3-hydroxy-2-naphthoic acid) or an L-97-1 di(3-hydroxy-2-naphthoic acid) salt (i.e., di(3-hydroxy-2-naphthoate salt); a 1:2 salt of L-97-1 and 3-hydroxy-2-naphthoic acid). Administration of an L-97-1 xinafoic acid salt or an L-97-1 3-hydroxy-2-naphthoic acid salt of the invention, particularly via pulmonary inhalation, promotes a desired therapeutic response. Such xinafoic acid salts or 3-hydroxy-2-naphthoic acid salts of L-97-1 can be delivered to a subject by, for example, pulmonary inhalation. Hydrated salts of the A₁ adenosine receptor antagonists of the invention are further provided and include, but are not limited to, 0.5 hydrate, monohydrate, 1.5 hydrate, dihydrate, 2.5 hydrate, and trihydrate forms.

The pharmaceutically acceptable salt of an A₁ adenosine receptor antagonist of the invention may be administered in combination with other therapeutic agents, either as a single pharmaceutical composition or as separate pharmaceutical compositions. Methods of the invention find use in treating subjects currently suffering from a respiratory disorder or in preventing the development of the disorder in subjects at risk for developing a respiratory condition. The methods also find use in preventing the acute onset of symptoms associated with the respiratory disorder in subjects presently afflicted with the disease.

### BRIEF DESCRIPTION OF THE DRAWINGS

FIG. 1 provides the results of adenosine provocation tests performed in allergic rabbits. The concentration of adenosine required to reduce lung compliance by 50% (PC₅₀) was determined at 15 minutes and 24 hours following L-97-1 administration by pulmonary inhalation. Experimental details are provided in Example 1.

### DETAILED DESCRIPTION OF THE INVENTION

The present invention is directed to compositions and methods for treating and preventing respiratory disorders, particularly diseases associated with reversible airway obstruction, including but not limited to asthma. The compositions of the invention comprise pharmaceutically acceptable salts of A₁ adenosine receptor antagonists. A₁ adenosine receptor antagonists of particular interest include those compounds described in U.S. Pat. Nos. 5,786,360 and 6,489,332, in co-pending U.S. application Ser. Nos. 10/780,296, entitled “A₁ Adenosine Receptor Antagonists,” filed Feb. 17, 2004, and 10/861,677, entitled “A₁ Adenosine Receptor Antagonists,” filed Jun. 4, 2004, and in co-pending International Application No. PCT/US2004/018171, entitled “A₁ Adenosine Receptor Antagonists,” filed Jun. 7, 2004, all of which are herein incorporated by reference in their entirety. In some embodiments, the A₁ adenosine receptor antagonist comprises a compound of formula (I): 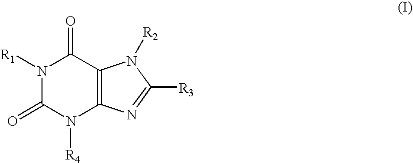
wherein R₁ is selected from the group consisting of C₁-C₈ alkyl;

R₂ is of the formula: 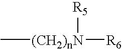
wherein n is an integer ranging from 1 to 8; R₅ is H or CH₃(CH₂)ₚ, wherein p is an integer ranging from 1 to 7; and R₆ is H; (CH₂)ₘH; or (CH₂)ₘOH, wherein m is an integer ranging from 1 to 8;

R₃ is:
—(CH₂)_{q}C₆H₄—R₇

wherein q is an integer ranging from 1 to 8; wherein R₇ is selected from the group consisting of H, OH, NH₂, R₉COOH, wherein R₉ is an alkylene or alkenylene group having 1 to 8 carbon atoms, and (CH₂)ₜOH, wherein t is an integer ranging from 1 to 8; and

R₄ is of the formula:

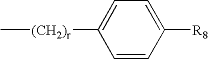

wherein R₈ is selected from the group consisting of H, NH₂, OH, (CH₂)_{f}NH₂ wherein f is an integer ranging from 1 to 8, (CH₂)ₙOH, wherein s is an integer ranging from 1 to 8, and R₁₀COOH, wherein R₁₀ is an alkylene or alkenylene group having 1 to 8 carbon atoms; and r is an integer ranging from 1 to 8. Methods for synthesizing the A₁ adenosine receptor antagonists of the invention are known in the art and are described in, for example, U.S. Pat. Nos. 5,786,360 and 6,489,332.

In a particular aspect of the invention, the A₁ adenosine receptor antagonist is 3-[2-(4-aminophenyl)ethyl]-8-benzyl-7-{2-[ethyl-(2-hydroxyethyl)amino]ethyl}-1-propyl-3,7-dihydropurine-2,6-dione, designated L-97-1, and comprises the compound of formula (I), wherein:

R₁ is C₃ alkyl;

R₂ is: 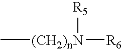
wherein n is 2; R₅ is CH₃(CH₂)ₚ, wherein p is 1; and R₆ is (CH₂)ₘOH, wherein m is 2;

R₃ is:
—(CH₂)_{q}C₆H₄—R₇
wherein q is 1; wherein R₇ is H; and

R₄ is of the formula: 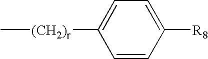
wherein R₈ is NH₂; and r is 2.

Although a number of pharmaceutically acceptable salts of A₁ adenosine receptor antagonists (e.g., L-97-1) can be produced, salts of these compounds that exhibit improved pulmonary inhalational drug characteristics are of particular interest. By “improved pulmonary inhalational drug characteristics” is intended that formulation of the A₁ adenosine receptor antagonist in a particular salt form produces a composition with reduced negative systemic side effects and a prolonged duration of action in the lung as a bronchodilator, anti-inflammatory, antimucolytic, antitussive, antifibrotic agent, antiangiogenesis agent, and/or enhancer of airway hydration, following administration by pulmonary inhalation. Because xinafoic acid salts are known to exhibit reduced oral absorption, as described herein above, xinafoic acid salts of A₁ adenosine receptor antagonists are of particular interest. Xinafoic acid (1-hydroxy-2-napthoic acid (Merck Index, 14^{th} Edition, 2006, 4834)), is an ionic salt that is largely water insoluble, thereby limiting oral absorption (and likely minimizing systemic effects) of a xinafoic acid salt of a compound following inhalational therapy. Xinafoic acid salts, such as salmeterol xinafoate, are known and have been synthesized in the art. See, for example, Merck Index, supra, and U.S. Pat. No. 4,992,474, both of which are herein incorporated by reference in their entirety. In certain embodiments, the composition comprises a xinafoic acid salt of an A₁ adenosine receptor antagonist, more particularly an L-97-1 xinafoic acid salt. The chemical structures of exemplary A₁ adenosine receptor antagonists as xinafoic acid salts, namely L-97-1 monoxinafoic acid salt (also alternately referred to herein as “L-97-1 monoxinafoate” or “L-97-1 monoxinafoate salt”) and L-97-1 dixinafoic acid salt (also alternately referred to herein as “L-97-1 dixinafoate” or “L-97-1 dixinafoate salt”) are set forth below:

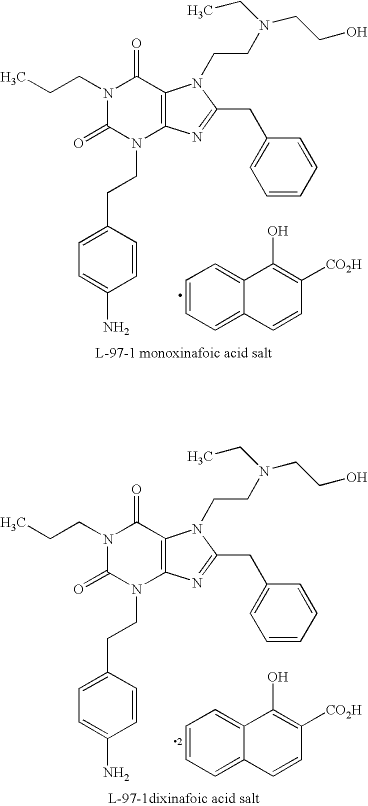

3-Hydroxy-2-napthoic acid (Merck Index, 14th Edition, 2006, 4835) also forms ionic salts that are largely water insoluble, thereby limiting oral absorption and likely minimizing systemic effects of a 3-hydroxy-2-naphthoic acid salt of a compound following inhalational therapy. 3-hydroxy-2-naphthoic acid salts, such as salmeterol 3-hydroxy-2-naphthoic acid salt, are known and have been synthesized in the art. See, for example, Merck Index, supra, and U.S. Pat. No. 4,992,474, both of which are herein incorporated by reference in their entirety. In certain embodiments, the composition comprises a 3-hydroxy-2-naphthoic acid salt of an A₁ adenosine receptor antagonist, more particularly an L-97-1 3-hydroxy-2-naphthoic acid salt. The chemical structures of exemplary L-97-1 3-hydroxy-2-naphthoic acid salts are set forth below:

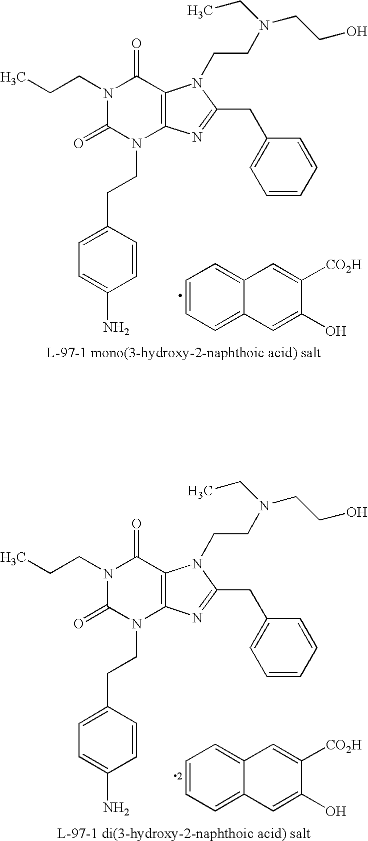

The A₁ adenosine receptor antagonists of the invention may form pharmaceutically acceptable salts with both organic and inorganic acids and bases. Exemplary weak organic acids for salt formation include but are not limited to acetic acid, beta-alanine, dl-alanine, D-alanine, L-alanine, formic acid, propanoic acid, butyric acid, palmetic acid, oleic acid, sebacic acid, cinnamic acid, adipic acid, citric acid, ascorbic acid (vitamin C), lactic acid, malic acid, maleic acid, fumaric acid, tartartic acid, dl-glutamic acid, D-glutamic acid, L-glutamic acid, dl-aspartic acid, D-aspartic acid, L-aspartic acid, glycine, succinic acid, glutaric acid, gluconic acid, benzoic acid, p-chlorobenzoic acid, p-hydroxybenzoic acid, p-methoxybenzoic acid, o-hydroxybenzoic acid (salicylic acid), 1-hydroxy-2-naphthoic acid (1-hydroxy-2-naphthalenecarboxylic acid, xinafoic acid), 3-hydroxy-2-naphthoic acid (3-hydroxy-2-naphthalenecarboxylic acid), and the like. Strong organic acids that may be used for salt formation include, for example, benzenesulfonic acid, p-toluenesulfonic acid, m-nitrobenzenesulfonic acid, methanesulfonic acid, ethanesulfonic acid, 1-naphthalenesulfonic acid, 2-naphthalenesulfonic acid, lauryl hydrogen sulfate, and the like. Examples of strong inorganic acids for salt formation include hydrochloric acid, hydrobromic acid, hydroiodic acid, sulfuric acid, phosphoric acid, nitric acid, sodium bisulfate, potassium bisulfate, sodium hydrogen phosphate, potassium hydrogen phosphate, boric acid, and the like. In a particular embodiment of the invention, an L-97-1 xinafoic acid salt is formed with 1-hydroxy-2-naphthoic acid (i.e., xinafoic acid; Merck Index, 14^{th} Edition, 2006, 4834). In another embodiment of the invention, an L-97-1 3-hydroxy-2-naphthoic acid salt is formed with 3-hydroxy-2-naphthoic acid (Merck Index, 14^{th} Edition, 2006, 4835).

Hydrated salts of the A₁ adenosine receptor antagonists of the invention are further provided and include, but are not limited to, 0.5 hydrate, monohydrate, 1.5 hydrate, dihydrate, 2.5 hydrate, or trihydrate forms.

Pharmaceutically acceptable salts of A₁ adenosine receptor antagonists of the invention, such as an L-97-1 xinafoic acid salt or an L-97-1 3-hydroxy-2-naphthoic acid salt, in a pharmaceutically acceptable carrier are further provided. The compositions find particular use in the methods disclosed herein. The compositions of the invention find use, for example, in preventing or ameliorating the nasal congestion, wheezing, coughing, shortness of breath, chest tightness, and other symptoms associated with respiratory disorders of the invention.

The methods of the invention are directed to treating or preventing a respiratory disorder. The methods of the invention comprise administering to a subject a therapeutically effective amount of a pharmaceutically acceptable salt of an A₁ adenosine receptor antagonist, more particularly an L-97-1 xinafoic acid salt or an L-97-1 3-hydroxy-2-naphthoic acid salt. Pharmaceutical compositions comprising a pharmaceutically acceptable salt of an A₁ adenosine receptor antagonist of the invention, such as an L-97-1 xinafoic acid salt or an L-97-1 3-hydroxy-2-naphthoic acid salt, and a second therapeutic agent are also provided. Methods of using these pharmaceutical compositions in the treatment and prevention of respiratory disorders are further disclosed.

The methods and compositions of the invention are useful in treating and preventing respiratory disorders. As used herein, “respiratory disorder” refers to a variety of disease states that involve the lungs and include but are not limited to allergic rhinitis, asthma, chronic bronchitis, chronic obstructive pulmonary disease, cystic fibrosis, adult respiratory distress syndrome, acute lung injury associated with septicemia or reperfusion organ injury, pulmonary fibrosis, bronchopulmonary dysplasia, emphysema, bronchiolitis obliterans (or bronchiolitis obliterans syndrome), and airway remodeling. One of skill in the art will appreciate the meaning of “respiratory disorder” and will recognize conditions encompassed by this term. “Respiratory disorder, “respiratory disease,” and “respiratory condition” are used interchangeably herein.

Administration of a composition of the invention may be for either treatment or prophylactic (i.e., preventative) purposes. “Treating” or “treatment” is herein defined as the administration of a therapeutically effective amount of a pharmaceutically acceptable salt of an A₁ adenosine receptor antagonist of the invention, more particularly an L-97-1 xinafoic acid salt or an L-97-1 3-hydroxy-2-naphthoic acid salt, where the subject has a respiratory condition or symptoms of a respiratory condition, where the purpose is to cure, heal, alleviate, relieve, alter, remedy, ameliorate, improve, or affect the respiratory condition or the symptoms of the respiratory condition. In certain aspects of the invention the L-97-1 xinafoic acid salt (e.g., a 0.5:1, 1:1, 1.5:1, or 2:1 L-97-1 xinafoic acid salt) or the L-97-1 3-hydroxy-2-naphthoic acid salt (e.g., a 0.5:1, 1:1, 1.5:1, or 2:1 L-97-1 3-hydroxy-2-naphthoic acid salt) is administered in combination with a second therapeutic agent is administered in combination with a second therapeutic agent. Such second “therapeutic agents” include, for example, antibiotics, anti-viral agents, anti-fungal agents, other bronchodilators, including beta-2 adrenergic receptor agonists, anti-cholinergics, anti-histamines, phosphodiesterase (PDE) inhibitors, particularly PDE-IV (PDE-4) inhibitors, leukotriene receptor antagonists, anti-inflammatory agents including but are not limited to glucocorticoids, cromolyn, and nonsteroidal anti-inflammatory drugs, mast cell stabilizers such as cromoglycate, surfactants, corticosteroids, such as beclomethasone dipropionate, fluticasone propionate, fluticasone furoate, P_{2X} purinoceptor antagonists, A_{2b} adenosine receptor antagonists, A₂ₐ adenosine receptor agonists, A₃ adenosine receptor agonists, other xanthines, A₁ adenosine receptor antagonists, A₃ adenosine receptor antagonists, anticytokines, 5-lipoxygenase inhibitors, platelet activating factor antagonists, thromboxane receptor antagonists, chemokine antagonists, such as VLA 4 antagonists and CCR-1 antagonist, neurokinin receptor antagonists, inhibitors of B cells, T cells, Leukocyte Selective Anti-inflammatory Drugs (LSAIDs), adhesion molecule antagonists, immunomodulators, such as lipopolysaccharide or Bacillus Calmette Guerain (BCG), immunosuppressants, adenosine production inhibitors, tryptase inhibitors, vaccines, complement inhibitors, kinase inhibitors, JAK kinase inhibitors, JAK 3 inhibitors, serine kinase inhibitors, and respiratory antisense oliogonucleotides (RASON). By “treatment” is also intended that a combination of the pharmaceutically acceptable salt of an A₁ adenosine receptor antagonist (e.g., an L-97-1 xinafoic acid salt or an L-97-1 3-hydroxy-2-naphthoic acid salt) and a second therapeutic agent are administered to the subject as part of a single pharmaceutical composition, or alternatively as part of individual pharmaceutical compositions, each comprising either the pharmaceutically acceptable salt of an A₁ adenosine receptor antagonist or the second therapeutic agent, where the subject has a respiratory condition or a symptom of a respiratory condition, where the purpose is to cure, heal, alleviate, relieve, alter, remedy, ameliorate, improve, or affect the condition or at least one of the symptoms of the respiratory condition.

Methods for preventing a respiratory condition are further disclosed. By “preventing a respiratory condition” is intended that a therapeutically effective amount of a pharmaceutically acceptable salt of an A₁ adenosine receptor antagonist, such as an L-97-1 xinafoic acid salt or an L-97-1 3-hydroxy-2-naphthoic acid salt, is administered to a subject at risk for developing a respiratory disorder in order to prevent the development of the disorder or is administered to a subject currently afflicted with a respiratory condition in order to prevent the acute onset of symptoms associated with the disorder or prevention of progression of the disorder. For example, in the case of asthma, an L-97-1 xinafoic acid salt or an L-97-1 3-hydroxy-2-naphthoic acid salt may be administered to an asthmatic patient in order to prevent the acute onset of symptoms such as nasal congestion, wheezing, coughing, shortness of breath, and chest tightness. The preventative methods of the invention also include combination therapies in which the pharmaceutically acceptable salt of the A₁ adenosine receptor antagonist (or a pharmaceutical composition comprising the salt in a pharmaceutically acceptable carrier) is administered in combination with another therapeutic agent, as described herein above.

In particular embodiments, the methods of the invention comprise using a combination therapy. The term “combination” is used in its broadest sense and means that a subject is treated with at least two therapeutic agents, more particularly a pharmaceutically acceptable salt of an A₁ adenosine receptor antagonist (e.g., an L-97-1 xinafoic acid salt or an L-97-1 3-hydroxy-2-naphthoic acid salt) and a second therapeutic agent, as defined herein above. The timing of administration of the salt of the A₁ adenosine receptor antagonist and the second therapeutic agent can be varied so long as the beneficial effects of the combination of these agents are achieved. The phrase “in combination with” refers to the administration of a pharmaceutically acceptable salt of an A₁ adenosine receptor antagonist, more particularly an L-97-1 xinafoic acid salt or an L-97-1 3-hydroxy-2-naphthoic acid salt, with a second therapeutic agent either simultaneously, sequentially, or a combination thereof. Therefore, a subject undergoing a combination therapy of the invention can receive the L-97-1 xinafoic acid salt or the L-97-1 3-hydroxy-2-naphthoic acid salt, and the second therapeutic agent at the same time (i.e., simultaneously) or at different times (i.e., sequentially, in either order, on the same day or on different days), so long as the therapeutic effect of the combination of both agents is achieved in the subject undergoing therapy. Where the L-97-1 xinafoic acid salt or the L-97-1 3-hydroxy-2-naphthoic acid salt and the second therapeutic agent are administered simultaneously, they can be administered as separate pharmaceutical compositions, each comprising either an L-97-1 xinafoic acid salt (or an L-97-1 3-hydroxy-2-naphthoic acid salt) or the second therapeutic agent, or can be administered as a single pharmaceutical composition comprising both agents.

The methods of the invention comprise administering to a subject a therapeutically effective amount of a pharmaceutically acceptable salt of an A₁ adenosine receptor antagonist. Any method for administering a composition to a subject may be used in the practice of the invention. Examples of possible routes of administration include pulmonary inhalation, parenteral, (e.g., intravenous (IV), intramuscular (IM), intradermal, subcutaneous (SC), or infusion), oral, nasal, transdermal (topical), transmucosal, and rectal administration. In certain aspects of the invention, particularly when the salt of an A₁ adenosine receptor antagonist comprises an L-97-1 xinafoic acid salt or an L-97-1 3-hydroxy-2-naphthoic acid salt, a therapeutically effective amount of the composition is delivered to the subject by pulmonary inhalation. By “therapeutically effective dose,” “therapeutically effective amount,” or “effective amount” is intended an amount of the pharmaceutically acceptable salt of an A₁ adenosine receptor antagonist, particularly a xinafoic acid salt or a 3-hydroxy-2-naphthoic acid salt, more particularly an L-97-1 xinafoic acid salt or an L-97-1 3-hydroxy-2-naphthoic acid salt, that brings about a positive therapeutic response with respect to treatment or prevention of a respiratory condition, as defined herein above. For example, a therapeutically effective amount of an L-97-1 xinafoic acid salt or an L-97-1 3-hydroxy-2-naphthoic acid salt may be an amount sufficient to prevent the acute onset of nasal congestion, wheezing, coughing, shortness of breath, difficulty breathing, and chest tightness in an asthmatic subject. In certain aspects of the invention, a therapeutically effective dose of an L-97-1 xinafoic acid salt or an L-97-1 3-hydroxy-2-naphthoic acid salt for administration by pulmonary inhalation is from about 2 picograms to about 200 micrograms per puff. In other embodiments, a therapeutically effective dose of an L-97-1 xinafoic acid salt or an L-97-1 3-hydroxy-2-naphthoic acid salt for intravenous administration is in the range from about 0.001 mg/kg to about 10 mg/kg. Determination of therapeutically effective amounts is well within the capability of those skilled in the art. The appropriate amount of a pharmaceutically acceptable salt of an A₁ adenosine receptor antagonist, for example, an L-97-1 xinafoic acid salt or an L-97-1 3-hydroxy-2-naphthoic acid salt, is readily determined by one of ordinary skill in the art without undue experimentation.

“Positive therapeutic response” refers to, for example, improving the condition of at least one of the symptoms of the respiratory disorder, preventing the worsening of at least one respiratory disorder-related symptom, or preventing or limiting the progression of the respiratory disorder. An improvement in at least one of the symptoms of the respiratory disorder can be assessed by a physician using routine laboratory tests, assessment of physiological data (e.g., pulmonary function testing, exercise tolerance assessment, such as exercise testing, arterial blood gas analysis, and determining oxygen saturation with pulse oximetry), chest radiograph and other radiological criteria, standard physical examination of the patient, bronchoscopy, measurements of levels of cytokines in the plasma, measurements of adenosine in the plasma or bronchoalveolar fluid, measurements of adenosine or nitric oxide in exhaled condensate, other measurements of inflammation and allergic disease, including levels of histamine and biomarkers of leukocytes, eosinophil, and mast cells or biomarkers of leukocyte, eosinophil, and mast cell function in plasma, bronchoalveolar fluid or exhaled condensates, or measurements of cytokines and biomarkers of leukocytes, specifically eosinophil and neutrophil function, in nasal secretions.

The decision to begin the methods for treating or preventing a respiratory disorder described herein in a subject will be made in accordance with assessment of the subject by a physician. For example, a physician may initiate the therapeutic methods of the invention when the subject is exhibiting clinical symptoms of the respiratory disorder or after the diagnosis of the disorder but prior to the onset of clinical symptoms. Symptoms of respiratory disorders, such as asthma, and methods for diagnosing them are well known in the art. A physician may also choose to initiate the therapeutic methods described herein for a patient at risk of developing the respiratory disorder prior to the appearance of clinical symptoms or a diagnosis of the condition. In other aspects of the invention, the methods may be used as a maintenance therapy to prevent the acute onset of symptoms of the respiratory disorder or progression of the disorder. For example, an L-97-1 xinafoic acid salt or an L-97-1 3-hydroxy-2-naphthoic acid salt may be administered to an asthmatic patient on a routine basis to prevent the onset of acute “attacks” of the respiratory disorder (e.g., nasal congestion, wheezing, coughing, difficulty breathing, and chest tightness) and progressive airway disease, e.g. airway remodeling, such as airway fibrosis, mucous gland hyperplasia, bronchial smooth muscle hypertrophy or angiogenesis.

A physician of ordinary skill in the art can determine when treatment for a respiratory disorder should be initiated and for how long the treatment should continue. Such treatment decisions may be supported by standard clinical laboratory results that monitor the clinical manifestations of the respiratory condition. The methods of the invention may be practiced by continuously or intermittently administering a therapeutically effective dose of the pharmaceutically acceptable salt of an A₁ adenosine receptor antagonist, alone or in combination with a second therapeutic agent, for as long as deemed efficacious for the treatment of the respiratory disorder. The decision to end therapy by the method of the invention may also be supported by standard clinical laboratory results indicating, for example, the disappearance of at least one of the clinical symptoms characteristic of the particular respiratory disorder. The therapy described herein may be restarted upon the return of respiratory condition.

The pharmaceutically acceptable salt of the A₁ adenosine receptor antagonist (e.g., an L-97-1 xinafoic acid salt or an L-97-1 3-hydroxy-2-naphthoic acid salt) is administered at a concentration that is therapeutically effective to treat or prevent a respiratory disorder. To accomplish this goal, the A₁ adenosine receptor antagonist salt may be formulated using a variety of acceptable excipients known in the art. A salt of an A₁ adenosine receptor antagonist, particularly a xinafoic acid salt or a 3-hydroxy-2-naphthoic acid salt, more particularly an L-97-1 xinafoic acid salt or an L-97-1 3-hydroxy-2-naphthoic acid salt, may be administered, for example, by pulmonary inhalation. Salts of an A₁ adenosine receptor antagonist may be administered, for example, by pulmonary inhalation or by intravenous, intraperitoneal, intramuscular, nasal, or subcutaneous injection. Methods to accomplish such administration are known to those of ordinary skill in the art. It may also be possible to obtain compositions which may be topically, sublingually, or orally administered, or which may be capable of transmission across mucous membranes.

Factors influencing the mode of administration and the appropriate amount of the pharmaceutically acceptable salt of an A₁ adenosine receptor antagonist of the invention, administered alone or in combination with a second therapeutic agent, include, but are not limited to, the severity of the disease, the history of the disease, and the age, height, weight, health, medical history (e.g., existence of other diseases such as diabetes, kidney or liver disease, and other drugs or treatments the patient is currently taking or has taken in the past), and physical condition of the individual undergoing therapy. Similarly, the amount of the therapeutic agents disclosed herein to be administered will be dependent upon the mode of administration and whether the subject will undergo a single dose or multiple doses of the pharmaceutically acceptable salt of an A₁ adenosine receptor antagonist. Generally, a higher dosage of is preferred with increasing weight of the patient undergoing therapy.

The treatment and prevention of respiratory disorders described herein can be accomplished with varying doses as well as dosage regimens. Treatment regimens will be based on doses and dosing schedules that maximize therapeutic effects. The therapeutically effective amount of a pharmaceutically acceptable salt of an A₁ adenosine receptor antagonist such as an L-97-1 xinafoic acid salt or an L-97-1 3-hydroxy-2-naphthoic acid salt can be readily determined by one of ordinary skill in the art without undue experimentation. In particular embodiments when combination therapies are performed, the therapeutically effective dose of a combination of a salt of an A₁ adenosine receptor antagonist and a second therapeutic agent may comprise doses of the individual agents that, when administered alone, would not be therapeutically effective or would be less therapeutically effective than when administered in combination with each other. For example, when an L-97-1 xinafoic acid salt or an L-97-1 3-hydroxy-2-naphthoic acid salt and a second therapeutic agent are administered in combination, a synergistic therapeutic effect may be observed. “Synergistic therapeutic effect” refers to a therapeutic effect observed with a combination of two or more therapies (in this case, the salt of the A₁ adenosine receptor antagonist and the second therapeutic agent) wherein the therapeutic effect on the respiratory disorder (as measured by any of a number of parameters) is greater than the sum of the respective individual therapeutic effects observed with the respective individual therapies. The combination of a pharmaceutically acceptable salt of an A₁ adenosine receptor antagonist, particularly a xinafoic acid salt or a 3-hydroxy-2-naphthoic acid salt, more particularly an L-97-1 xinafoic acid salt or an L-97-1 3-hydroxy-2-naphthoic acid salt, and a second therapeutic agent may produce a synergistic effect that permits a reduction in the dosages of these agents and an improvement of the clinical outcome of the subject being treated. A reduced dose of the salt of an A₁ adenosine receptor antagonist and the second therapeutic agent may in turn reduce unwanted side effects associated with each agent.

In some embodiments of the invention, the method comprises administration of multiple doses of a pharmaceutically acceptable salt of an A₁ adenosine receptor antagonist. The method may comprise administration of 1, 2, 3, 4, 5, 6, 7, 8, 9, 10, 15, 20, 25, 30, 35, 40, or more therapeutically effective doses of a pharmaceutical composition comprising a pharmaceutically acceptable salt of an A₁ adenosine receptor antagonist, including an L-97-1 xinafoic acid salt or an L-97-1 3-hydroxy-2-naphthoic acid salt, either alone or in combination with a second therapeutic agent. The frequency and duration of administration of multiple doses of the pharmaceutical compositions can be readily determined by one of skill in the art without undue experimentation. Moreover, treatment of a subject with a therapeutically effective amount of an L-97-1 xinafoic acid salt or an L-97-1 3-hydroxy-2-naphthoic acid salt or another salt of an adenosine receptor antagonist of the invention can include a single treatment or can include a series of treatments. In particular aspects of the invention, an L-97-1 xinafoic acid salt or an L-97-1 3-hydroxy-2-naphthoic acid salt may be administered on a routine basis (e.g., daily or multiple times per day) to a subject, for example, an asthmatic patient, in order to prevent the acute onset of symptoms associated with the respiratory disease (e.g., nasal congestion, wheezing, coughing, shortness of breath, difficulty breathing, and chest tightness). It will also be appreciated that the effective dosage of a pharmaceutically acceptable salt of an A₁ adenosine receptor antagonist, particularly a xinafoic acid salt or a 3-hydroxy-2-naphthoic acid salt, more particularly an L-97-1 xinafoic acid salt or an L-97-1 3-hydroxy-2-naphthoic acid salt, may increase or decrease over the course of a particular treatment. Necessary changes in dosage may become apparent from the results of standard medical tests known in the art.

The pharmaceutically acceptable salt of an A₁ adenosine receptor antagonist is typically provided to the subject by a standard technique within a pharmaceutically acceptable buffer; for example, sterile saline, sterile buffered water, propylene glycol, combinations of the foregoing, etc. When the methods of the invention comprise the administration of a pharmaceutically acceptable salt of an A₁ adenosine receptor antagonist in combination with a second therapeutic agent, the salt of the A₁ adenosine receptor antagonist and the second therapeutic agent can be formulated in separate pharmaceutical compositions, or can be formulated within a single pharmaceutical composition for simultaneous administration. Methods for preparing parenterally administrable agents are described in Remington's Pharmaceutical Sciences (20^{th} ed.; Philadelphia College of Pharmacy and Science, Philadelphia, Pa., 2000), herein incorporated by reference.

The pharmaceutically acceptable salts of the A₁ adenosine receptor antagonists of the invention, particularly an L-97-1 xinafoic acid salt or an L-97-1 3-hydroxy-2-naphthoic acid salt, can be administered alone, but may also be administered in admixture with a pharmaceutically acceptable carrier selected with regard to the intended route of administration and pharmaceutical practice. Thus, a pharmaceutical composition of the invention is formulated to be compatible with its intended route of administration. Examples of possible routes of administration include pulmonary inhalation, parenteral, (e.g., intravenous (IV), intramuscular (IM), intradermal, subcutaneous (SC), or infusion), oral, nasal, sublingual, transdermal (topical), transmucosal, and rectal administration. When the pharmaceutically acceptable salt of an A₁ adenosine receptor antagonist comprises a xinafoic acid salt or a 3-hydroxy-2-naphthoic acid salt, particularly an L-97-1 xinafoic acid salt or an L-97-1 3-hydroxy-2-naphthoic acid salt, the route of administration is optimally not an oral delivery of the composition. A “pharmaceutically acceptable carrier” refers to a carrier that is conventionally used in the art to facilitate the storage, administration, or the therapeutic effect of the active ingredient. A suitable carrier may also reduce any undesirable side effects of the salt of the A₁ adenosine receptor antagonist. It should not produce significant local or systemic adverse effects in recipients at the dosages and concentrations employed for treatment. Methods for formulating pharmaceutical compositions are generally known in the art. A thorough discussion of formulation and selection of pharmaceutical acceptable carriers, stabilizers, and isomolytes can be found in Remington 's Pharmaceutical Sciences (20^{th} ed.; Philadelphia College of Pharmacy and Science, Philadelphia, Pa., 2000), herein incorporated by reference.

In particular embodiments, the pharmaceutically acceptable salt of an A₁ adenosine receptor antagonist, for example, an L-97-1 xinafoic acid salt or an L-97-1 3-hydroxy-2-naphthoic acid salt, or a pharmaceutical composition thereof, is formulated for administration to a subject via pulmonary inhalation. By “pulmonary inhalation” is intended the composition of the invention is directly administered to the lung by delivering the composition in an aerosol or other suitable preparation from a delivery device into the oral cavity of the subject as the subject inhales through the oral cavity. By “aerosol” is intended a suspension of solid or liquid particles in flowing air or other physiologically acceptable gas stream. Other suitable preparations include, but are not limited to, mist, vapor, or spray preparations so long as the particles comprising the protein composition are delivered in a size range consistent with that described for a dry powder form of the composition as defined herein. Pulmonary inhalation could also be accomplished by other suitable methods known to those skilled in the art. These may include liquid instillation using a suitable device or other such methods. Pulmonary inhalation results in deposition of the inhaled composition in the across the nasal epithelium, small airways (bronchi), or alveoli of the subject's lungs. Once deposited, the composition may be absorbed, passively or actively, across the bronchial or alveolar epithelium.

Pulmonary administration of a composition of the invention such as an L-97-1 xinafoic acid salt or an L-97-1 3-hydroxy-2-naphthoic acid salt requires dispensing of the composition from a delivery device into the oral cavity of a subject during inhalation. For purposes of the present invention, compositions comprising pharmaceutically acceptable salts of an A₁ adenosine receptor antagonist (or pharmaceutical compositions thereof) are administered via inhalation of an aerosol or other suitable preparation that is obtained from an aqueous or nonaqueous solution or suspension form, or a solid or dry powder form of the composition, depending upon the delivery device used. Such delivery devices are well known in the art and include, but are not limited to, nebulizers, metered-dose inhalers, and dry powder inhalers, or any other appropriate delivery mechanisms that allow for dispensing of a composition as an aqueous or nonaqueous solution or suspension or as a solid or dry powder form. By “aqueous” is intended a composition prepared with, containing, or dissolved in water, including mixtures wherein water is the predominating substance in the mixture. A predominating substance is present in a greater quantity than another component of the mixture. By “nonaqueous” is intended a composition prepared with, containing, or dissolved in a substance other than water or mixtures wherein water is not the predominating substance in the mixture. By “solution” is intended a homogeneous preparation of two or more substances, which may be solids, liquids, gases, or combinations thereof. By “suspension” is intended a mixture of substances such that one or more insoluble substances are homogeneously dispersed in another predominating substance.

For purposes of the present invention, the terms “solid” and “dry powder” are used interchangeably. By “solid” or “dry powder” form of a composition is intended the composition has been dried to a finely divided powder having a moisture content below about 10% by weight, usually below about 5% by weight, and preferably below about 3% by weight. This dry powder form of the composition consists of particles comprising the L-97-1 xinafoic acid salt or the L-97-1 3-hydroxy-2-naphthoic acid salt or other pharmaceutically acceptable salt of an A₁ adenosine receptor antagonist of the invention. Preferred particle sizes are less than about 10.0 μm mean diameter, more preferably less than about 7.0 μm, even more preferably about less than about 6.0 μm, even more preferably in the range of 0.1 to 5.0 μm, most preferably in the range of about 1.0 to about 5.0 μm mean diameter.

Thus, a liquid composition comprising an L-97-1 xinafoic acid salt or an L-97-1 3-hydroxy-2-naphthoic acid salt or another pharmaceutically acceptable salt of an A₁ adenosine receptor antagonist intended for use in the methods of the present invention may either be used as a liquid solution or suspension in the delivery device or first be processed into a dry powder form using lyophilization or spray-drying techniques well known in the art. Where a liquid solution or suspension is used in the delivery device, a nebulizer, a metered dose inhaler, or other suitable delivery device delivers, in a single or multiple fractional dose, by pulmonary inhalation a therapeutically effective amount of the composition is delivered to the subject's nose or lungs as droplets having the same particle size range noted above for the dry powder form. The liquid solution or suspension of the composition may be used with physiologically appropriate stabilizing agents, excipients, bulking agents, surfactants, or combinations thereof, as discussed below. Examples of suitable excipients are well known in the art and include, but are not limited to, buffers, viscosity modifiers, or other therapeutically inactive but functional additives.

Where the liquid composition is lyophilized prior to use in the delivery methods of the invention, the lyophilized composition is milled to obtain the finely divided dry powder consisting of particles within the desired size range noted above. Where spray-drying is used to obtain a dry powder form of the liquid composition, the process is carried out under conditions that result in a substantially amorphous finely divided dry powder consisting of particles within the desired size range noted above. Similarly, if the starting composition is already in a lyophilized form, the composition can be milled to obtain the dry powder form for subsequent preparation as an aerosol or other preparation suitable for pulmonary inhalation. Where the starting composition is in its spray-dried form, the composition has preferably been prepared such that it is already in a dry powder form having the appropriate particle size for dispensing as an aqueous or nonaqueous solution or suspension or dry powder form in accordance with the pulmonary administration methods of the invention. For methods of preparing dry powder forms of compositions, see, for example, WO 96/32149, WO 97/41833, WO 98/29096, and U.S. Pat. Nos. 5,976,574, 5,985,248, and 6,001,336; all of which are herein incorporated by reference in their entirety.

The resulting dry powder form of the composition is then placed within an appropriate delivery device for subsequent preparation as an aerosol or other suitable preparation that is delivered to the subject via pulmonary inhalation. Where the dry powder form of the composition is to be prepared and dispensed as an aqueous or nonaqueous solution or suspension, a metered-dose inhaler, or other appropriate delivery device is used. A therapeutically effective amount of the dry powder form of the composition is administered in an aerosol or other preparation suitable for pulmonary inhalation. The amount of dry powder form of the composition placed within the delivery device is sufficient to allow for delivery of a therapeutically effective amount of the composition to the subject by inhalation. Thus, the amount of dry powder form to be placed in the delivery device will compensate for possible losses to the device during storage and delivery of the dry powder form of the composition. Following placement of the dry powder form within a delivery device, the properly sized particles as noted above are suspended in an aerosol propellant. The pressurized nonaqueous suspension is then released from the delivery device into the air passage of the subject while inhaling. The delivery device delivers, in a single or multiple fractional doses, by pulmonary inhalation a therapeutically effective amount of the composition to the subject's lungs. The aerosol propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoromethane, dichlorodifluoro-methane, dichlorotetrafluoroethanol, and 1,1,1,2-tetrafluoroethane, or combinations thereof. A surfactant may be added to the pharmaceutical composition to reduce adhesion of the protein-containing dry powder to the walls of the delivery device from which the aerosol is dispensed. Suitable surfactants for this intended use include, but are not limited to, sorbitan trioleate, soya lecithin, and oleic acid. Devices suitable for pulmonary delivery of a dry powder form of a composition as a nonaqueous suspension are commercially available. Examples of such devices include the Ventolin metered-dose inhaler (GlaxoSmithKline Inc., Research Triangle Park, N.C.) and the Intal Inhaler (Fisons, Corp., Bedford, Mass.). See also the aerosol delivery devices described in U.S. Pat. Nos. 5,522,378, 5,775,320, 5,934,272 and 5,960,792, herein incorporated by reference.

Where the solid or dry powder form of the composition is to be delivered as a dry powder form, a dry powder inhaler or other appropriate delivery device is preferably used. The dry powder form of the composition is preferably prepared as a dry powder aerosol by dispersion in a flowing air or other physiologically acceptable gas stream in a conventional manner. Examples of commercially available dry powder inhalers suitable for use in accordance with the methods herein include the Spinhaler powder inhaler (Fisons Corp., Bedford, Mass.) and the Ventolin Rotahaler (GlaxoSmithKline, Inc., Research Triangle Park, N.C.). See also the dry powder delivery devices described in WO 93/00951, WO 96/09085, WO 96/32152, and U.S. Pat. Nos. 5,458,135, 5,785,049, and 5,993,783, herein incorporated by reference.

The dry powder form of the composition comprising an L-97-1 xinafoic acid salt or an L-97-1 3-hydroxy-2-naphthoic acid salt (or other pharmaceutically acceptable salts of an A₁ adenosine receptor antagonist) can be reconstituted to an aqueous solution for subsequent delivery as an aqueous solution aerosol using a nebulizer, a metered dose inhaler, or other suitable delivery device. In the case of a nebulizer, the aqueous solution held within a fluid reservoir is converted into an aqueous spray, only a small portion of which leaves the nebulizer for delivery to the subject at any given time. The remaining spray drains back into a fluid reservoir within the nebulizer, where it is aerosolized again into an aqueous spray. This process is repeated until the fluid reservoir is completely dispensed or until administration of the aerosolized spray is terminated. Such nebulizers are commercially available and include, for example, the Ultravent nebulizer (Mallinckrodt Inc., St. Louis, Mo.) and the Acorn II nebulizer (Marquest Medical Products, Englewood, Colo.). See also the nebulizer described in WO 93/00951, and the device for delivering aerosolized aqueous formulations described in U.S. Pat. No. 5,544,646; herein incorporated by reference.

Methods and devices for administering compositions via pulmonary inhalation and for producing particles suitable for such administration are disclosed in the art. See, for example, U.S. Pat. Nos. 6,221,338, 6,475,523, 6,521,260, 6,582,678, 6,941,948, 6,948,496, 6,989,155; U.S. Patent Application Publication Nos. 2003/0170183, 2003/0202944, 2005/0013862, 2005/0152849, 2005/0158394, 2005/0205083, and 2006/0029552; all of which are herein incorporated by reference in their entirety.

In other embodiments, a salt of an A₁ adenosine receptor antagonist of the invention may be administered intranasally. Intranasal or nasal administration of a composition of the invention such as an L-97-1 xinafoic acid salt or an L-97-1 3-hydroxy-2-naphthoic acid salt requires dispensing of the composition from a delivery device into the nasal cavity of a subject. Intranasal administration represents a convenient alternative route for administration, particularly for self-administration of an A₁ adenosine receptor antagonist salt of the invention. Intranasal formulations may generally be provided in liquid or in dry powder forms. Satisfactory intranasal formulations must be sufficiently stable (i.e., chemically and physically) to be consistently dispensed in accurate metered doses. Accordingly, the active ingredient must be compatible with the excipients used in the formulation and should not aggregate in a manner which would result in a loss of accurate dose delivery, for example by precipitation from a liquid formulation or by caking of a powder formulation. To maximize retention of an intranasal formulation within the nasal passages of a patient after administration, particularly of a liquid formulation, it is desirable to deliver the unit dosage of active ingredient within a relatively small delivery volume. This may necessitate the use of high concentrations of medicament and highly soluble active ingredients are therefore advantageous. Clearly, an active ingredient must also be presented in a form which is readily absorbed through the nasal mucosa but which is unassociated with any adverse effects such as irritancy.

A salt of an A₁ adenosine receptor antagonist of the invention will generally be administered in the form of a solution. Such solutions will generally be aqueous and prepared with, for example, sterile or pyrogen-free water alone or water and a physiologically acceptable co-solvent (e.g., ethanol, propylene glycol, polyethylene glycols such as PEG 400). These solutions may additionally contain other excipients such as preservatives, buffering agents, isotonicity-adjusting agents (e.g., sodium chloride), viscosity enhancing agents, absorption enhancers, flavoring agents (e.g., aromatic flavoring agents such as menthol, eucalyptol, camphor, and methyl salicylate), and sweetening agents (e.g., saccharin). Preferably solutions according to the invention for intranasal administration will be sterile and free from preservatives. Methods for preparing formulations suitable for intranasal administration are routine in the art.

Typically solutions for intranasal administration are applied directly to the nasal cavity by conventional means, for example, with a dropper, pipette, or spray. Devices for intranasal delivery of a composition to a subject are well known in the art. See, for example, U.S. Pat. Nos. 6,186,141 and 5,705,520. The formulations may be provided in single or multi-dose form. In the latter case, a means of dose metering is desirably provided. In the case of a dropper or pipette this may be achieved by the patient administering an appropriate, predetermined volume of the solution. In the case of a spray this may be achieved for example by means of a metering atomizing spray pump. A salt of the present invention may conveniently be presented in unit dose form.

Intranasal administration may also be achieved by means of an aerosol formulation in which the compound is provided in a pressurized pack with a suitable propellant such as a chlorofluorocarbon (CFC), for example dichlorodifluoromethane, trichlorofluoromethane or dichlorotetrafluoroethane, a hydrofluorocarbon (HFC) for example 1,1,1,2-tetrafluoroethane or 1,1,1,2,3,3,3-heptafluoropropane, carbon dioxide or other suitable gas. The dose of drug may be controlled by provision of a metered valve.

Although in certain aspects of the invention the xinafoic acid salts or the 3-hydroxy-2-naphthoic acid salts of the A₁ adenosine receptor antagonists are optimally delivered to a subject via pulmonary inhalation or intranasal delivery, pharmaceutically acceptable salts of A₁ adenosine receptor antagonists may be administered to a subject in accordance with any method known in the art. When a composition of the invention is administered by intravenous, intradermal, or subcutaneous injection, the composition is in the form of a pyrogen-free, parenterally acceptable aqueous solution. The preparation of such parenterally acceptable solutions, having due regard to pH, isotonicity, stability, and the like, is well within the skill in the art. Solutions or suspensions used for parenteral, intradermal, subcutaneous, or intravenous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid disodium salt; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes, multiple dose vials made of glass or plastic, or plastic bags of intravenous solutions (e.g. dextrose, ringers lactate or normal saline).

The pharmaceutically acceptable salts of the A₁ adenosine receptor antagonists of the invention can be formulated for parenteral administration by injection, e.g., by bolus injection or continuous infusion. Formulations for injection can be presented in unit dosage form, e.g., in ampoules or in multidose containers, with an added preservative. The compositions can take such forms as suspensions, solutions, or emulsions in oily or aqueous vehicles, and can contain formulatory agents such as suspending, stabilizing, and/or dispersing agents.

For oral administration, the compositions of the invention can be formulated by combining a salt of a compound of formula (I) with pharmaceutically acceptable carriers well known in the art. Such carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding a compound of formula (I) with a solid excipient, optionally grinding or milling a resulting mixture, and processing the mixture of granules, after adding suitable auxiliaries, if desired, to obtain tablets or dragee cores. Suitable excipients include, for example, fillers and cellulose preparations. If desired, disintegrating agents can be added.

The exact formulation, route of administration, and dosage of the compositions of the invention can be chosen by the individual physician in view of the patient's condition. Dosage amount and dosing intervals can be adjusted individually to provide plasma levels of the salt of the A₁ adenosine receptor antagonist, particularly xinafoic acid salts or 3-hydroxy-2-naphthoic acid salts, more particularly an L-97-1 xinafoic acid salt or an L-97-1 3-hydroxy-2-naphthoic acid salt, that are sufficient to maintain positive therapeutic effects.

One of skill in the art will appreciate that the methods for treating and preventing respiratory disorders disclosed herein can be combined with any other therapy for treating or preventing respiratory disorders. Such therapies include but are not limited to antibiotics, anti-viral agents, anti-fungal agents, other bronchodilators, including beta-2 adrenergic receptor agonists, anti-cholinergics, anti-histamines, phosphodiesterase (PDE) inhibitors, particularly PDE-IV inhibitors, leukotriene receptor antagonists, anti-inflammatory agents including but not limited to glucocorticoids, cromolyn, and nonsteroidal anti-inflammatory drugs, mast cell stabilizers such as cromoglycate, surfactants, corticosteroids such as beclomethasone dipropionate, fluticasone propionate, fluticasone furoate, P_{2X} purinoceptor antagonists, A_{2b} adenosine receptor antagonists, A₂ₐ adenosine receptor agonists, A₃ adenosine receptor agonists, other xanthines, A₁ adenosine receptor antagonists, A₃ adenosine receptor antagonists, anticytokines, 5-lipoxygenase inhibitors, platelet activating factor antagonists, thromboxane receptor antagonists, chemokine antagonists, such as VLA 4 antagonists and CCR-1 antagonist, neurokinin receptor antagonists, inhibitors of B cells, T cells, Leukocyte Selective Anti-inflammatory Drugs (LSAIDs), adhesion molecule antagonists, immunomodulators, such as lipopolysaccharide or Bacillus Calmette Guerain (BCG), immunosuppressants, adenosine production inhibitors, tryptase inhibitors, vaccines, complement inhibitors, kinase inhibitors, JAK kinase inhibitors, JAK 3 inhibitors, serine kinase inhibitors, and respiratory antisense oliogonucleotides (RASON).

The present invention also provides for the use of a pharmaceutically acceptable salt of an A₁ adenosine receptor antagonist, particularly an L-97-1 xinafoic acid salt or an L-97-1 3-hydroxy-2-naphthoic acid salt, in the manufacture of a medicament for treating or preventing a respiratory disorder. “Treating” or “treatment” in the context of the use of a medicament comprising a pharmaceutically acceptable salt of an A₁ adenosine receptor antagonist (e.g., an L-97-1 xinafoic acid salt or an L-97-1 3-hydroxy-2-naphthoic acid salt) described herein is defined as the application or administration of the medicament to a subject, where the subject has a respiratory condition, a symptom associated with a respiratory condition, or a predisposition toward development of a respiratory disorder, where the purpose is to cure, heal, alleviate, relieve, alter, remedy, ameliorate, improve, or affect the respiratory disorder, any associated symptoms of the respiratory disorder, the predisposition toward the development of the respiratory disorder, or progression of the respiratory disorder.

The present invention also provides for the use of a synergistic combination of a pharmaceutically acceptable salt of an A₁ adenosine receptor antagonist, particularly a xinafoic acid salt or a 3-hydroxy-2-naphthoic acid salt, more particularly an L-97-1 xinafoic acid salt or an L-97-1 3-hydroxy-2-naphthoic acid salt, in the manufacture of a medicament for treating a subject for a respiratory disorder, wherein the medicament is coordinated with treatment using a second therapeutic agent, as defined herein above. By “synergistic combination” is intended that the medicament comprising an amount of the salt of the A₁ adenosine receptor antagonist provides for a synergistic therapeutic effect when the medicament is coordinated with treatment using a second therapeutic agent as set forth herein above. As indicated above, “synergistic therapeutic effect” refers to a therapeutic effect observed with a combination of two or more therapies (in this case, the pharmaceutically acceptable salt of an A₁ adenosine receptor antagonist, such as an L-97-1 xinafoic acid salt or an L-97-1 3-hydroxy-2-naphthoic acid salt, and a second therapeutic agent) wherein the therapeutic effect (as measured by any of a number of parameters) is greater than the sum of the respective individual therapeutic effects observed with the respective individual therapies.

The methods of treatment of the present invention are not intended to be limited to particular subjects. A variety of subjects, particularly mammals, are contemplated. Subjects of interest include but are not limited to humans, dogs, cats, horses, pigs, cows, and rodents. In particular embodiments, the subject is a human.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The following examples are offered by way of illustration and not by way of limitation.

### EXPERIMENTAL

### Example 1

### L-97-1 Blocks Adenosine Provocation Test Following Aerosol Administration

L-97-1 was administered as an inhalational treatment before the inhalational administration of adenosine in allergic rabbits. All animals were anesthetized and intubated. All treatments were administered via an endotracheal tube with an ultrasonic nebulizer that produces aerosol droplets (80% are less than 5 μm). Dynamic compliance (C_{dyn}) was calculated from tidal volume and the difference in transpulmonary pressure (P_{TP}) at zero flow (V). Total lung resistance (R_{T}) was calculated as the ratio of P_{TP} and V at midtidal lung volumes; these measurements are made automatically with the Buxco Respiratory Analyzer. Baseline bronchial hyperresponsiveness (BHR) was established in the allergic rabbits by challenging the rabbits with increasing concentrations of aerosolized histamine. The PC₅₀ for histamine (concentration of histamine to reduce lung compliance by 50%) was determined. The animals were allowed to recover from the effects of histamine for at least one day.

Animals were challenged with increasing concentrations of aerosolized adenosine to determine the PC₅₀ of adenosine (controls, n=9). These same animals were allowed to recover from the effects of adenosine and were treated with aerosolized L-97-1 (5 mg/ml) for 2 minutes. Following L-97-1, they were challenged with increasing concentrations of adenosine 15 minutes (n=5) or 24 hours later (n=4). The PC₅₀ of adenosine in the presence of L-97-1 was determined at 15 minutes and at 24 hours.

Results

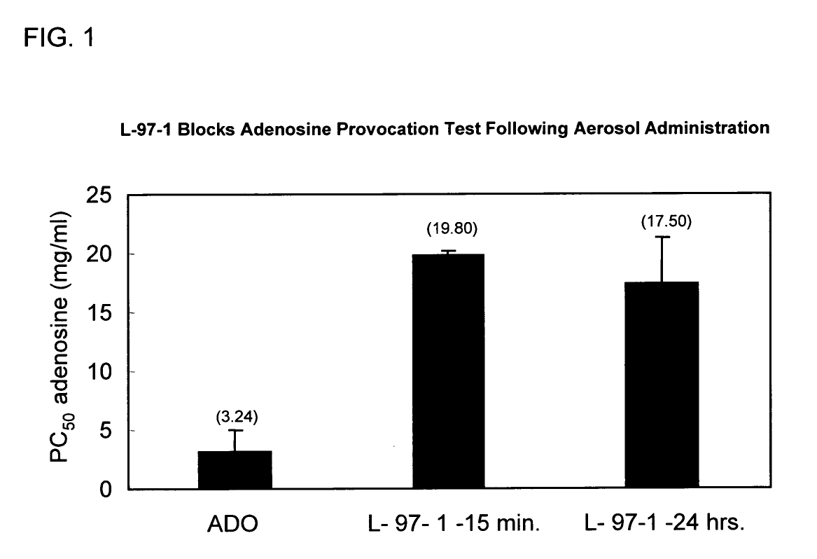

The PC₅₀ for histamine (baseline BHR, n=5) was 0.77±0.28 mg/ml; the PC₅₀ for adenosine (controls, n=9) is 3.24±1.79 mg/ml. The PC₅₀ for adenosine following administration of L-97-1 at 15 minutes (L-97-1 15 min, n=5) was 19.8±0.32, and the PC₅₀ for adenosine following L-97-1 administration at 24 hours (L-97-1 24 hrs, n=4) was 17.5±3.75. The results are summarized in FIG. 1.

### Example 2

### Synthesis of L-97-1 Monoxinafoic Acid Salt (L-97-1 Monoxinafoate)

L-97-1 monoxinafoic acid salt is synthesized according to the process illustrated below:

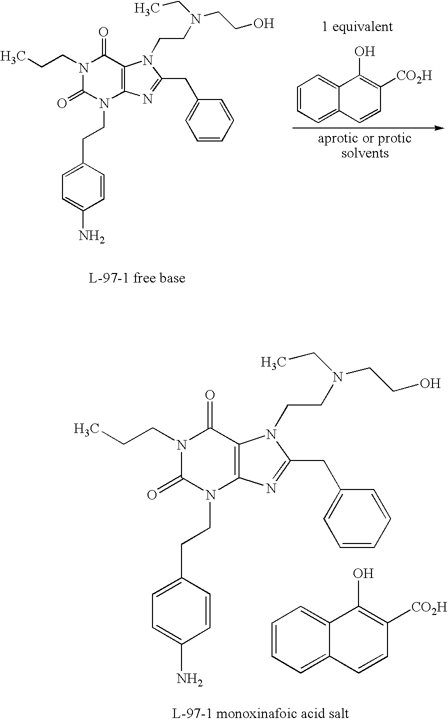

A mixture of 518 mg (1.0 mmole) of L-97-1 free base and 188 mg (1.0 mmole) of xinafoic acid (1-hydroxy-2-naphthoic acid) is stirred at room temperature in a suspension with 5.0 ml of deionized water for 24 hours to produce the finely divided insoluble hydrated L-97-1 monoxinafoic acid salt. The off-white solid is collected by filtration and air dried to yield the hydrated form of L-97-1 monoxinafoic acid salt. Depending upon the amount of drying, the 0.5 hydrate, the monohydrate, the 1.5 hydrate, the dihydrate, the 2.5 hydrate, or the trihydrate salt can be formed. Any of these materials can also be heated at 30-40° C. for 1-2 days under 0.1-1 mm vacuum to produce the anhydrate form of L-97-1 monoxinafoic acid salt.

### Example 3

### Synthesis of L-97-1 0.5 Xinafoic Acid Salt (L-97-1 0.5 Xinafoate)

A mixture of 518 mg (1.0 mmole) of L-97-1 free base and 94 mg (0.5 mmole) of xinafoic acid (1-hydroxy-2-naphthoic acid) is stirred at room temperature in a suspension with 5.0 ml of deionized water for 24 hours to produce the finely divided insoluble hydrated L-97-1 0.5 xinafoic acid salt. The off-white solid can be collected by filtration and air dried to yield the hydrated form of L-97-1 0.5 xinafoic acid salt. Depending upon the amount of drying, the 0.5 hydrate, the monohydrate, the 1.5 hydrate, the dihydrate, the 2.5 hydrate, or the trihydrate salt can be formed. Any of these materials can also be heated at 30-40° C. for 1-2 days under 0.1-1 mm vacuum to produce the anhydrate form of L-97-1 0.5 xinafoic acid salt.

### Example 4

### Synthesis of L-97-1 1.5 Xinafoic Acid Salt (L-97-1 1.5 Xinafoate)

A mixture of 259 mg (0.5 mmole) of L-97-1 free base and 141 mg (0.75 mmole) of xinafoic acid (1-hydroxy-2-naphthoic acid) is stirred at room temperature in a suspension with 5.0 ml of deionized water for 24 hours to produce the finely divided insoluble hydrated L-97-1 1.5 xinafoic acid salt. The off-white solid is collected by filtration and air dried to yield the hydrated form of L-97-1 1.5 xinafoic acid salt. Depending upon the amount of drying, the 0.5 hydrate, the monohydrate, the 1.5 hydrate, the dihydrate, the 2.5 hydrate, or the trihydrate salt can be formed. Any of these materials can also be heated at 30-40° C. for 1-2 days under 0.1-1 mm vacuum to produce the anhydrate form of L-97-1 1.5 xinafoic acid salt.

### Example 5

### Synthesis of L-97-1 Dixinafoic Acid Salt (L-97-1 Dixinafoate)

L-97-1 dixinafoic acid salt is synthesized according to the process illustrated below:

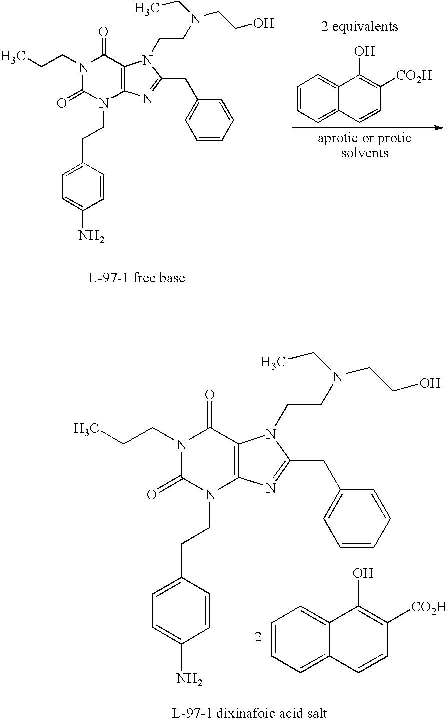

A mixture of 518 mg (1.0 mmole) of L-97-1 free base and 376 mg (2.0 mmole) of xinafoic acid (1-hydroxy-2-naphthoic acid) is stirred at room temperature in a suspension with 5.0 ml of deionized water for 24 hours to produce the finely divided insoluble hydrated L-97-1 dixinafoic acid salt. The off-white solid is collected by filtration and air dried to yield the hydrated form of L-97-1 dixinafoic acid salt. Depending upon the amount of drying, the 0.5 hydrate, the monohydrate, the 1.5 hydrate, the dihydrate, the 2.5 hydrate, or the trihydrate salt can be formed. Any of these materials can also be heated at 30-40° C. for 1-2 days under 0.1-1 mm vacuum to produce the anhydrate form of L-97-1 dixinafoic acid salt.

### Example 6

### Synthesis of L-97-1 Mono(3-Hydroxy-2-Naphthoic Acid) Salt

L-97-1 mono(3-hydroxy-2-naphthoic acid) salt is synthesized according to the process illustrated below:

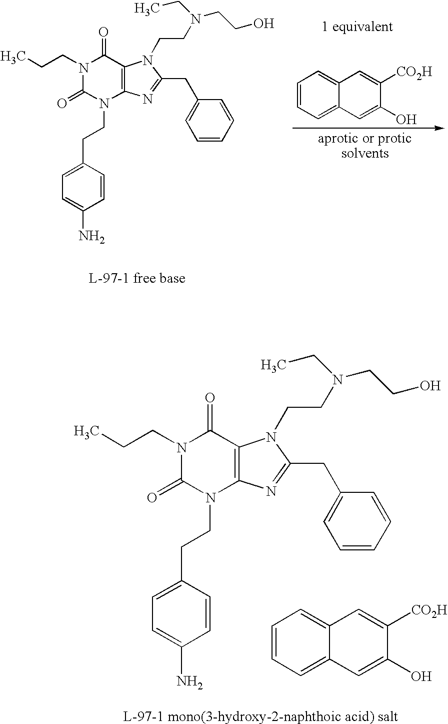

By the method of Example 2, a mixture of 518 mg (1.0 mmole) of L-97-1 free base and 188 mg (1.0 mmole) of 3-hydroxy-2-naphthoic acid is stirred at room temperature in a suspension with 5.0 ml of deionized water for 24 hours to produce the finely divided insoluble hydrated L-97-1 mono(3-hydroxy-2-naphthoic acid) salt. The off-white solid is collected by filtration and air dried to yield the hydrated form of L-97-1 mono(3-hydroxy-2-naphthoic acid) salt. Depending upon the amount of drying, the 0.5 hydrate, the monohydrate, the 1.5 hydrate, the dihydrate, the 2.5 hydrate, or the trihydrate salt can be formed. Any of these materials can also be heated at 30-40° C. for 1-2 days under 0.1-1 mm vacuum to produce the anhydrate form of L-97-1 mono(3-hydroxy-2-naphthoic acid) salt.

In a similar manner, the 0.5 (3-hydroxy-2-naphthoic acid) salt, the 1.5 (3-hydroxy-2-naphthoic acid) salt, and the di(3-hydroxy-2-naphthoic acid) salt forms of L-97-1 can be synthesized in either hydrated or anhydrate forms.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A salt of an A₁ adenosine receptor antagonist, wherein the salt is a 1-hydroxy-2-naphthoic acid salt or a 3-hydroxy-2-naphthoic acid salt, and wherein the A₁ adenosine receptor antagonist comprises a compound of formula (I): 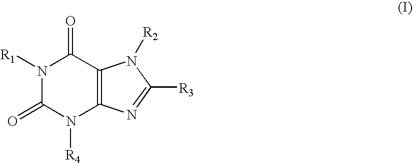
wherein R₁ is selected from the group consisting of C₁-C₈ alkyl;
R₂ is of the formula: 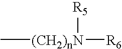
wherein n is an integer ranging from 1 to 8; R₅ is H or CH₃(CH₂)ₚ, wherein p is an integer ranging from 1 to 7; and R₆ is H; (CH₂)ₘH; or (CH₂)ₘOH, wherein m is an integer ranging from 1 to 8;
R₃ is:
—(CH₂)_{q}C₆H₄—R₇
wherein q is an integer ranging from 1 to 8; wherein R₇ is selected from the group consisting of H, OH, NH₂, R₉COOH, wherein R₉ is an alkylene or alkenylene group having 1 to 8 carbon atoms, and (CH₂)ₜOH, wherein t is an integer ranging from 1 to 8; and
R₄ is of the formula: 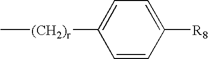
wherein R₈ is selected from the group consisting of H, NH₂, OH, (CH₂)_{f}NH₂ wherein f is an integer ranging from 1 to 8, (CH₂)ₙOH, wherein s is an integer ranging from 1 to 8, and R₁₀COOH, wherein R₁₀ is an alkylene or alkenylene group having 1 to 8 carbon atoms; and r is an integer ranging from 1 to 8.

2. The salt of claim 1, wherein the A₁ adenosine receptor antagonist comprises the compound of formula (I), wherein:
R₁ is C₃ alkyl;
R₂ is: 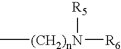
wherein n is 2; R₅ is CH₃(CH₂)ₚ, wherein p is 1; and R₆ is (CH₂)ₘOH, wherein m is 2;
R₃ is:
—(CH₂)_{q}C₆H₄—R₇
wherein q is 1; wherein R₇ is H; and
R₄ is of the formula: 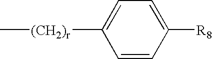
wherein R₈ is NH₂; and r is 2.

3. A method of preventing or treating a respiratory disorder in a subject, the method comprising administering to the subject a therapeutically effective amount of the salt according to claim 1.

4. The method of claim 3, wherein the respiratory condition is selected from the group consisting of allergic rhinitis, asthma, chronic bronchitis, chronic obstructive pulmonary disease, cystic fibrosis, adult respiratory distress syndrome, acute lung injury associated with septicemia or reperfusion organ injury, pulmonary fibrosis, bronchopulmonary dysplasia, emphysema, bronchiolitis obliterans (or bronchiolitis obliterans syndrome), and airway remodeling.

5. The method of claim 4, wherein the respiratory condition is allergic rhinitis or asthma.

6. The method of claim 5, wherein the respiratory condition is asthma.

7. The method of claim 3, wherein the subject is a human patient.

8. The method of claim 3 further comprising administration of a second therapeutic agent.

9. The method of claim 8, wherein the second therapeutic agent is selected from the group consisting of antibiotics, anti-viral agents, anti-fungal agents, bronchodilators, beta-2 adrenergic receptor agonists, anti-cholinergics, anti-histamines, phosphodiesterase (PDE) inhibitors, PDE-IV (PDE-4) inhibitors, leukotriene receptor antagonists, anti-inflammatory agents, glucocorticoids, cromolyn, nonsteroidal anti-inflammatory drugs, mast cell stabilizers, cromoglycate, surfactants, steroids, corticosteroids, beclomethasone dipropionate, fluticasone propionate, fluticasone furoate, P_{2X} purinoceptor antagonists, xanthines, A_{2b} adenosine receptor antagonists, A₂ₐ adenosine receptor agonists, A₃ adenosine receptor agonists, A₁ adenosine receptor antagonists, A₃ adenosine receptor antagonists, anticytokines, 5-lipoxygenase inhibitors, platelet activating factor antagonists, thromboxane receptor antagonists, chemokine antagonists, VLA 4 antagonists, CCR-1 antagonist, neurokinin receptor antagonists, inhibitors of B cells, T cells, Leukocyte Selective Anti-inflammatory Drugs (LSAIDs), adhesion molecule antagonists, immunomodulators, lipopolysaccharide, Bacillus Calmette Guerain (BCG), immunosuppressants, adenosine production inhibitors, tryptase inhibitors, vaccines, complement inhibitors, kinase inhibitors, JAK kinase inhibitors, JAK 3 inhibitors, serine kinase inhibitors, and respiratory antisense oliogonucleotides (RASON).

10. The method of claim 8, wherein the salt of the A₁ adenosine receptor antagonist and the second therapeutic agent are administered sequentially or simultaneously.

11. The method of claim 3, wherein the salt of the A₁ adenosine receptor antagonist is administered by a method selected from the group consisting of pulmonary inhalation, nasal, sublingual, intravenous, intramuscular, intradermal, and subcutaneous administration.

12. The method of claim 11, wherein the salt of the A₁ adenosine receptor antagonist is administered by pulmonary inhalation.

13. The method of claim 3, wherein the method comprises preventing the acute onset of symptoms in a subject currently afflicted with the respiratory disorder.

14. The method of claim 13, wherein the subject is afflicted with allergic rhinitis or asthma.

15. The method of claim 14, wherein the subject is afflicted with asthma.

16. The method of claim 13, wherein the symptoms comprise nasal congestion, wheezing, coughing, shortness of breath, or chest tightness.

17. A pharmaceutical composition comprising the salt according to claim 1 in a pharmaceutically acceptable carrier.

18. A method of preventing or treating a respiratory disorder in a subject, the method comprising administering to the subject a therapeutically effective amount of the pharmaceutical composition according to claim 17.

19. The method of claim 18, wherein the subject is a human patient.

20. The method of claim 18, wherein the method comprises preventing the acute onset of symptoms in a subject currently afflicted with the respiratory disorder.

21. The method of claim 20, wherein the subject is afflicted with allergic rhinitis or asthma.

22. The method of claim 21, wherein the subject is afflicted with asthma.

23. The method of claim 20, wherein the acute onset of symptoms comprises nasal congestion, wheezing, coughing, shortness of breath, or chest tightness.

24. The salt of claim 1, wherein the salt is selected from the group consisting of L-97-1 0.5 xinafoic acid salt, L-97-1 monoxinafoic acid salt, L-97-1 1.5 xinafoic acid salt, and L-97-1 dixinafoic acid salt.

25. A hydrate of the salt of claim 24, wherein the hydrate is a 0.5 hydrate, a monohydrate, a 1.5 hydrate, a dihydrate, a 2.5 hydrate, or a trihydrate.

26. The salt of claim 1, wherein the salt is selected from the group consisting of L-97-1 0.5 (3-hydroxy-2-naphthoic acid) salt, L-97-1 mono(3-hydroxy-2-naphthoic acid) salt, L-97-1 1.5 (3-hydroxy-2-naphthoic acid) salt, and L-97-1 di(3-hydroxy-2-naphthoic acid) salt.

27. A hydrate of the salt of claim 26, wherein the hydrate is a 0.5 hydrate, a monohydrate, a 1.5 hydrate, a dihydrate, a 2.5 hydrate, or a trihydrate.
